(19) United States
(12) Patent Application Publication
(10) Pub. No.: US 2023/0210626 A1
(43) Pub. Date: Jul. 6, 2023

(54) METHOD AND DEVICE FOR DOCUMENTING THE USE OF AT LEAST ONE IMPLANT WHICH IS USED IN A SURGERY AND/OR THE LOCALIZATION THEREOF

(71) Applicant: Wolfgang AUF, Graz (AT)

(72) Inventor: Wolfgang AUF, Graz (AT)

(21) Appl. No.: 17/922,010

(22) PCT Filed: Apr. 7, 2021
(86) PCT No.: PCT/AT2021/060118

§ 371 Date: Oct. 28, 2022

(30) Foreign Application Priority Data
Apr. 30, 2020 (AT) A 50372/2020

(51) Int. Cl. A61B 90/00; A61B 90/94; A61B 90/30; A61B 50/33; H04N 23/56; G06V 20/50; G06V 10/764; G06V 10/75; H04N 5/262; H04N 23/695; G06T 7/00; G16H 40/20

(52) U.S. Cl. CPC A61B 90/08; A61B 90/94; A61B 90/361; A61B 90/30; A61B 50/33; H04N 23/56; G06V 20/50; G06V 10/764; G06V 10/751; H04N 5/2628; H04N 23/695; G06T 7/0014; G16H 40/20; A61B 2090/0805; G06V 2201/03; G06T 2207/30052; G06T 2207/10016

(57) ABSTRACT

A method and device for documenting use of at least one implant used in a surgery and/or for the localization thereof. The implant can be provided for a surgery and used in the surgery. The method includes: a) providing a surgical set having a plurality of implants; b) capturing a first sequence of images of the plurality of implants of the surgical set using a device; c) analyzing the sequence of images of the plurality of implants in order to identify each individual implant; d) optionally outputting a signal when one and/or each implant has been identified; e) capturing a second sequence of images of the plurality of implants of the surgical set using the device after a surgery in order to ascertain missing implants; f) classifying a missing implant as used in surgery.

The invention relates to a method for documenting a use of at least one implant used in a surgery and/or for the localization thereof, wherein the implant can be provided for a surgery and used in the surgery.

Furthermore, the invention relates to a device for documenting and/or localizing a use of at least one implant used in a surgery, wherein the implant can be provided for a surgery and used in the surgery.

Implants are frequently used in the treatment of bone fractures or the remedying of anatomical misalignments of bones, for example an ulnar shortening, as well as other surgical procedures. The implants, for example bone plates, intramedullary nails, Kirschner wires, or the like, are normally provided together with suitable fastening means, in particular screws such as fixed-angle screws, in what are referred to as surgical sets. In addition to a selection of different bone plates, a surgical set of this type comprises, for example, a plurality of screws and other implants or small implant parts, so that the appropriate parts can be removed as needed during a surgery and can be used and implanted in the surgery.

Because the implants are used on or in the human or animal body and remain therein at least temporarily, a careful documentation is necessary so that appropriate measures can be taken, for example, if, due to their composition, individual implants can no longer be used or, in the event that they have already been used, must be removed and replaced. It is noted solely by way of example that certain implant alloys, for example the magnesium alloy WE 43, contain considerable additions of rare earth metals, the long-term effect of which when an implant breaks down in the body is not yet precisely known and has not yet been investigated in detail.

A further case in which the origin of the implant is of critical significance is a damage event when the implant, for example a bone plate, fails. In this case, it may be necessary, for example for questions of liability law, to be able to precisely ascertain the manufacturer and/or the batch of a manufacturer from which the implant originates.

In order to be able to ensure the corresponding traceability of the origin of an implant or the localization thereof in later use, it is often required that documentation of which implants or parts of a surgical set are used occurs in a surgery. For this purpose, a surgical assistant must perform the necessary documentation, which is normally time-intensive and can also cause considerable problems. This applies in particular to small implant screws, since these can have a comparatively small design. This makes a reading of a batch number and/or part number that are necessary for the documentation considerably difficult. Often times, this costly and laborious step of reading numbers on the implants leads to a documentation not being carried out in a proper manner. As a result, it is thus neither possible to trace which parts of a surgical set were used, nor is it possible to subsequently trace where an implant originates from.

To address this problem, attempts have already been made to provide bone screws and other implants, including small implant screws in particular, separately in plastic bags, wherein the batch number and, where necessary, other numbers such as the part number are noted on the plastic bag. However, this inevitably leads to the need to provide a plurality of bags in a more complicated surgery. In addition, the remaining parts of the surgical set still need to be identified and documented if the entirety of all implants used is to be recorded.

From the prior art, other additional methods have also become known, for example from WO 2016/074672 A2, which discloses a method for automatically documenting the use of surgical implants, wherein an image of a set of surgical implants provided for a surgery is produced on a screen and a user interface is then generated so that command buttons are associated with the supplied implants, after which a removal of implants is subsequently documented. However, this method is relatively costly, since an association via the graphical user interface must occur for every surgical implant or individual part. In addition, each manual intervention constitutes a potential source of error.

Other methods according to the prior art function, in addition to a visual recording or alternatively thereto, using a recording of the weight of a surgical set. However, individual parts cannot be reliably identified therefrom.

This is addressed by the invention. The object of the invention is to further develop a method of the type named at the outset such that, in a simple and automated manner, it is possible to ascertain which implants were used in a surgery so that the implants can then also be localized.

A further object of the invention is to specify a device suitable for this purpose.

The method-related object of the invention is attained if, in a method of the type named at the outset, the following method steps are provided:

a) providing a surgical set having a plurality of implants:

b) capturing a first sequence of images of the plurality of implants of the surgical set using a device;

c) analyzing the sequence of images of the plurality of implants in order to identify each individual implant;

d) optionally outputting a signal when one and/or each implant has been identified;

e) capturing a second sequence of images of the plurality of implants of the surgical set using the device after a surgery in order to ascertain missing implants;

f) classifying a missing implant as used in surgery.

An advantage obtained with the invention is in particular that, through multiple method steps, each simple in its own right, it is possible to reliably ascertain which implants have been used during a surgery. Those implants which are no longer located in the surgical set have necessarily been used in the surgery. These used implants can therefore be uniquely associated with the person who was treated.

An analysis of the sequence of images of the implants can occur using image recognition. It can thereby also be provided that implants are already stored or saved in a dataset and are matched therewith. For identifying an implant, however, it is already sufficient if the batch number and/or part number can be read out from the images.

In this relation, and also in the remaining context of the invention, implant is to be understood broadly and includes that portion of a surgical set which can be used in or on the human or animal body, in particular bone plates, various nails, but also screws and other small parts that are necessary for fastening bone plates to the bone, for example.

A uniqueness of the association of the implants is ensured in that the supplied surgical set is screened using a device, wherein a first sequence of images of the plurality of implants is captured. This sequence of images of the plurality of implants is matched, in particular using image recognition, in order to identify each individual implant or the type of an implant, in particular with the aid of the batch number and/or article number. A surgery cannot begin until each individual implant has been identified. If, during the capture of the sequence of images, it emerges that one or more implants are not able to be identified, a corresponding screening can be repeated until all implants are identified. A different sequence of images can thereby also be captured, so that different images are available for the evaluation and, if necessary, for a matching with a dataset. This is possible, for example, if the unit for capturing a sequence of the plurality of implants is positioned and/or moved at a different angle relative to the surgical set. The surgical set can also be positionally altered if individual implants have not yet been recognized. Also possible, of course, is a visual check of the surgical set, for example if it is not possible to identify all parts, for instance because non-implant objects, such as swabs, surgical instruments, or the like, were inadvertently placed in the surgical set. It can be provided that foreign objects of this type are classified as such by a user input, for example by manual selection on a touchscreen. No additional sequences of images then need to be captured for foreign objects of this type. An analogous procedure can occur if an implant cannot be identified. In this case, a user can mark the implant as unusable for the surgery, in particular via a touchscreen.

After the surgery, a further capture takes place, this time of a second sequence of images of the plurality of implants. Based on the difference from the first sequence of images of the plurality of implants, the missing contingent of those implants which have since been used in the surgery results. The missing implants had previously been uniquely identified with regard to their origin by batch number and/or part number with the aid of the evaluation of the first sequence of images of the plurality of implants, and have then been used on or in the human body being treated. The origin and position of the implant(s) used have thus been uniquely determined. The implant can also still be associated many years later, for example if an undesired implant failure occurs or implants need to be exchanged due to new research findings. To generate the difference, and thus determine the implants missing from the surgical set and used in the surgery, it is sufficient during the evaluation of the second sequence of images to determine the implant spaces no longer occupied.

Even though the method according to the invention is applied to any desired implants, such as hip or knee prostheses, stents, or intramedullary nails, or to other large implants, it proves effective particularly when small implants such as screws or the like are also used, since, based on experience, a reading of the identification features (part number and/or batch number or log number) in the case of small implants in particular causes the difficulties mentioned above.

In order to carry out the method rapidly, it is beneficial if in step b) the surgical set is traveled over, in particular traveled over in an automated manner, by image capturing equipment of the device. For this purpose, the image capturing equipment can be mounted such that it can be moved in a plane. Since the surgical set is normally embodied as a container in which a screen is inserted with the implants attached thereto or positioned thereon, in particular implants positioned in a plane of the screen, a parallel traveling-over in a parallel x,y plane over the surgical set can be sufficient. The image capturing equipment is then moved over the surgical set in a grid pattern in the x,y plane until all implants have been captured. For this purpose, the optical axis of the image capturing equipment is positioned to be perpendicular to the x,y plane so that the image capturing equipment is oriented downwards towards the surgical set. However, it is also possible that the image capturing equipment is additionally mounted such that it can be pivoted out of the x,y plane at a predetermined angle, for example up to 45°, from this position oriented exactly perpendicular to the x,y plane, and is correspondingly pivoted so that the individual implants can be captured not only in a 90° position, but also laterally. The information content for an image recognition and/or a matching with the dataset thus increases, since the implants are also covered from the side. Furthermore, it is possible, once again additionally or alternatively, that the image capturing equipment can, in addition to the capture in a grid pattern in an x,y plane, also be rotated about the axis oriented perpendicularly to the x,y plane for this purpose. Particularly if a tilting out of the x,y plane at an angle of ±45°, for example, is also possible, there then results a maximum information content concerning the appearance of the implant as well as the identification numbers placed thereon, such as a batch number and/or part number.

Because a unique matching with the aid of the captured images can in particular depend on the ability of numbers of the implants (batch number or log number and/or part number) to be captured and accurately recognized, it is advantageous if an enlarged image is outputted by the image capturing equipment. For this purpose, it can be provided that suitable optical equipment, for example a lens, is upstream of the image capturing equipment. The optical equipment such as a lens can also be an integral component of the image capturing equipment.

As previously mentioned, the image capturing equipment can essentially be guided over the surgical set in a plane. Even though an orientation for a corresponding scanning or a screening is preferably such that a grid plane is parallel to the plane in which the implants are positioned, a grid plane can in principle also occur at a slant such that the grid plane forms an angle with the positioning plane of the implants. This can be provided, for example, if implants with markedly different sizes are positioned in the surgical set, with the size of the implants increasing from small to large. In this case, it can be advisable to tilt the grid plane relative to the plane of the surgical set so that the surface of the implants always remains in the focus of the image capturing equipment, without the focus thereof needing to be readjusted.

It can be provided that the device is embodied such that it can be placed in a precisely fitting manner onto a surface of the surgical set, in particular such that a form fit is produced. The device comprises in this case a correspondingly fitting housing that can be placed onto the surgical set. The units necessary for the image capture are then arranged inside of the housing. A touchscreen with an evaluation unit can be mounted or integrated on the top side of the device. The method is then simplified for a user, since the device only needs to be placed onto the surgical set, after which the image capture and the evaluation thereof can occur. Errors that are caused by incorrect operation, are thereby prevented.

For particular precision, however, it can also be provided that the focus of the image capturing equipment is readjusted for each implant being captured, in order to obtain an optimal sequence of images. This can also take place in an automated manner.

It has been shown that it is beneficial if, during the capture of the sequence of images, the surgical set is illuminated at least in the region of one implant using light from a light source during the capture of the sequence of images in step b). In this manner, the result of the image capture and, in further consequence, therefore of a possible matching with a dataset can be improved. The light source can in particular be a light-emitting diode. An illumination using at least one light source can also occur in step e), wherein this can be the same light source that is also used in step b). It is expedient if the light source is moved simultaneously with the image capturing equipment so that consistent lighting conditions are ensured during an image capture.

The identification in step c) can be carried out via a batch number and/or part number read out from the images. In principle, however, it is also possible that the implants are identified solely with the aid of their structure. In the ideal case, an association or a matching takes place both via batch number and/or part number and also—in order to check and to further ensure the uniqueness—via the structure of the implant. For a structural matching, a further dataset is possibly provided with which a recognition and identification are performed following image recognition by a comparison with stored structures for implants.

The analysis and the possibly performed matching with a dataset can thereby occur after the capture of a sequence of the plurality of implants, that is, once the surgical set or the implants therein have been fully captured. The analysis and, where provided, the matching can, however, also take place dynamically to the effect that the matching with a dataset already occurs during the scanning of the surgical set and implants using a database. It is thus possible, for example, that each implant is immediately matched with the dataset and the image capturing equipment only moves again once the implant has been uniquely identified by means of the matching. In the context of the present application, dataset means any basis that, after a comparison with the evaluated sequence of images, is suitable for characterizing an implant as uniquely identified. In the simplest case, the dataset merely constitutes information about the manufacturer of the implants. The matching then involves, for example, the read-out batch numbers and/or part numbers with the manufacturer. Once these have been clearly read out, the implant is uniquely determined via the manufacturer and the batch number and/or part number thereof. Even simple digits and/or letters as a basis for the recognition of batch numbers and/or part numbers can be considered a dataset.

It can be provided that the identification in step c) includes an identification of structural features. As a result, an overdetermination is essentially ensured, since in addition to the batch numbers and/or part numbers, a structural matching is also present as an additional validation.

Accordingly, a batch number and/or part number and/or structural features can be determined from captured images and matched with a database.

It is expedient that the steps b) and c) are repeated if not all implants are identified. Since it cannot be ruled out that individual implants are defective and/or missing and/or non-implant parts are located in the surgical set, it can also be provided that in this case, for example via a graphical interface, individual non-identifiable parts or implants are skipped after an input by a user. In this case, it can be provided that, following the surgery, it must once again be confirmed that the unidentified, and therefore also unused, non-implant part (defective implant or foreign part in general) is still present, in order to ensure that said foreign part was not used.

Normally, a surgical set is provided with a screen, wherein the implants are arranged on the screen and/or attached thereto. It can also be provided that an audible and/or a visual signal is outputted when one and/or all implants have been uniquely identified. Via the output of audible and/or visual signals, for example via a graphical interface, the present stage of the matching or identification of implants can be rapidly indicated to the user. In particular, it can be indicated that the surgical set can be used for a surgery since all implants have been identified.

The other object of the invention is attained if a device of the type named at the outset comprises image capturing equipment for capturing a sequence of images of implants of a surgical set, evaluation equipment, and optionally a data carrier on which a dataset is stored, wherein the sequence of images can be evaluated using the evaluation equipment in order to identify implants, and a storage unit via which the localization of an implant can be depicted.

With a device according to the invention, it can be very easily ascertained which implants were used in a surgery. The implants used must necessarily be located in or on the human or animal body that was operated on. Via the unique association of the implants, it is thus possible to track down at a later point the place of origin, the manufacturer, and also the batch, so that in a damage event, for example, a unique association with manufacturing processes, compositions, or the like is possible. A dataset can thereby be provided for a matching. In a very simple form, the dataset comprises, for example, structural features of implants and/or numbers and digits, which are compared with the sequence of images of the implants using image recognition, in order to thus enable an identification of the implants.

Preferably, a movement mechanism is provided with which the image capturing equipment can be moved. The image capturing equipment can in particular be movable in an x,y plane. This x,y plane can run parallel to a plane in which the implants are arranged in the surgical set. Normally, then, the x,y plane is horizontally oriented. Other degrees of motion can be provided. For example, the image capturing equipment, the optical axis of which is typically oriented perpendicularly to an x,y plane running horizontally, which plane can in turn be oriented parallel to a plane of the surgical set in or on which the implants are arranged, can be pivotable out of said x,y plane and/or rotatable about its own optical axis in the x,y plane. This makes it possible to inspect the implants located in the surgical set not only perpendicularly from above, but also to a certain extent laterally, whereby the information content, and therefore the quality, of a possible unique association is improved or increased.

The image capturing equipment can be movable in a plane using the movement mechanism. For this purpose, a motor, in particular a servomotor or a different electric motor, can expediently be provided, which motor moves the image capturing equipment. The movement of the image capturing equipment can thereby be correlated with the identification of individual implants so that a movement only occurs, for example, if the implant currently being inspected has been uniquely identified or matched, or alternatively if it has been manually confirmed by a user that capturing can continue since no identification could be made.

Furthermore, a light source for illuminating the implants during the capture of the sequence of images can be provided. With an illumination, a higher quality of the images results for the subsequent image processing.

The device is particularly preferably embodied with a housing which surrounds all elements that are provided for the image capturing, and which is preferably embodied on the bottom side such that the device can be placed onto the surgical set in a form fit. On the top side, a unit or a device for evaluation and operation can be arranged on the device. For documentation, the device then only needs to be placed onto the surgical set and the image capturing equipment arranged inside the housing travels over the implants. All components necessary for this purpose, such as motors, light sources, or the like, are arranged inside of the housing. Only an operating device is located on the top side or constitutes or is integrated in said side.

Additional features, advantages, and effects of the invention follow from the exemplary embodiment illustrated below. In the drawings which are thereby referenced:

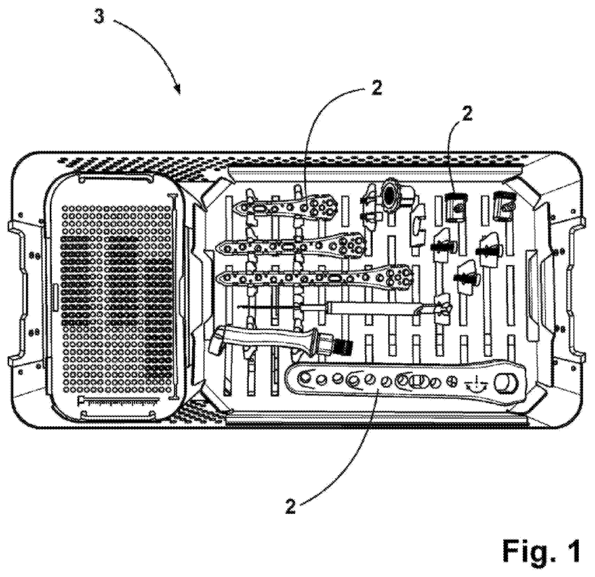

FIG. 1 shows a surgical set having a plurality of implants;

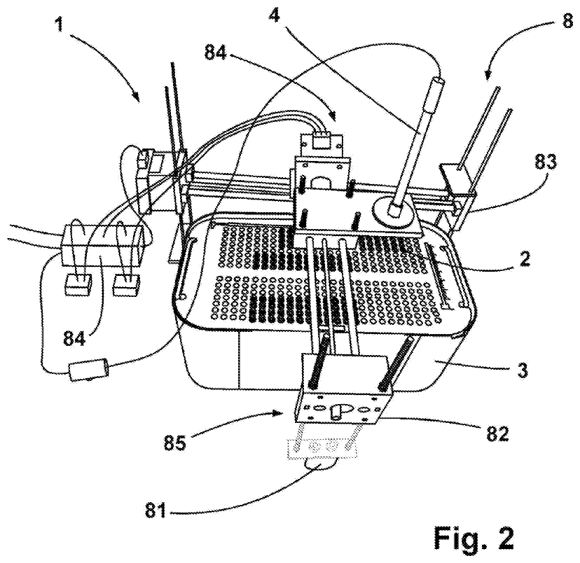

FIG. 2 shows a device according to the invention in a first perspective view;

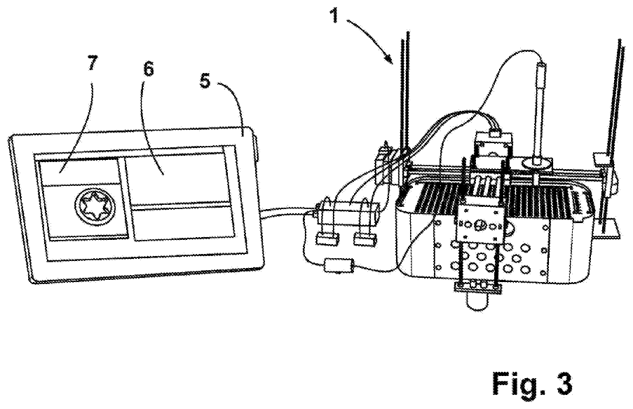

FIG. 3 shows the device according to the invention from FIG. 2 in another perspective view;

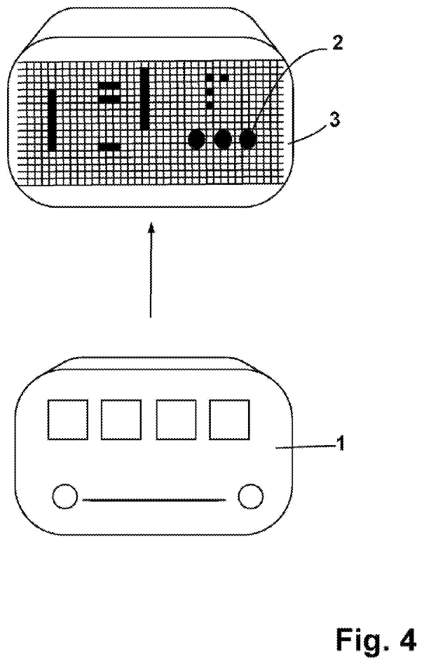

FIG. 4 shows a schematic illustration of a device according to the invention.

In FIG. 1, a surgical set 3 having a plurality of implants 2 is illustrated. The surgical set 3 comprises a container open at the top with an inserted screen on which a plurality of implants 2 is positioned and, if necessary, fixed in place.

A surgical set 3 according to FIG. 1 is supplied to the operating doctor(s) in a surgery. During the surgery, the implants 2 necessary for the specific surgery are then removed, although only a portion of the implants 2 is required, depending on what is needed for the surgery. In order to ascertain, given the plurality of implants 2 provided, those which were implanted, a documentation is necessary during the surgery. Without documentation, it can no longer be reliably ascertained afterwards which implants 2 were used given the plurality of implants 2, since the surgical set 3 is restocked after the surgery and, in the stocked state, is once again used in a surgery.

In FIG. 2 and FIG. 3, a device 1 according to the invention is illustrated, wherein the measuring construction is emphasized. In terms of size, the device according to the invention is designed such that a surgical set 3 together with the implants 2 arranged therein can be positioned inside of the device 1, or the device 1 can be placed onto the surgical set 3 in a precisely fitting manner. The device 1 comprises image capturing equipment 4 that is embodied as an endoscope in the example. The image capturing equipment 4 is oriented such that the optical axis thereof is perpendicular to a screen of the surgical set 3 on which the implants 2 are arranged. The image capturing equipment 4 is thus perpendicular to a plane in which the implants are arranged.

The image capturing equipment 4 is mounted on a transport unit of the device 1. With the transport unit, the image capturing equipment 4 can in particular be moved in an x,y plane that is parallel to the plane in which the implants 2 are arranged. It is thereby possible to travel over the entire x,y plane. As a result, it is possible to capture the screen or the implants 2 of the surgical set 3 completely in each case. A sequence of images that are evaluated in a manner which will be described below is thereby generated.

In the present example according to FIG. 2 and FIG. 3, the image capturing equipment 4 is rigidly mounted on a platform. However, it can also be provided that the image capturing equipment 4 is mounted on the platform or another base in an articulated manner so that the image capturing equipment 4 can be pivoted about the x,y plane at a predetermined angle, for example ±45°. It can furthermore be provided that the image capturing equipment 4 is alternatively or additionally mounted such that it can also be rotated in the x,y plane. This is then expedient if the optical axis of the image capturing equipment is not perpendicular to the x,y plane and/or no circular images are to be captured.

The construction according to FIG. 2 and FIG. 3 furthermore comprises a movement mechanism 8 that includes multiple parts. In the exemplary embodiment, the movement mechanism 8 is designed such that a stationary lateral frame 83 is provided on which the platform with the image capturing equipment 4 is hinged. The lateral frame 83 is designed such that the hinging of the platform together with image capturing equipment 4 is mounted such that it can be displaced in the x direction. For this purpose, a motor, not illustrated in greater detail, is provided with which the movement unit 84 can be moved back and forth in the x direction, whereby the platform with the image capturing equipment 4 is moved in the x direction. The platform is thereby mounted on rods which connect to the unit 84. Opposite of the unit 84 is a further unit 85 that serves as a counter bearing. The two units 84, 85 are connected to one another via three rods. The unit 85 is thereby embodied with a bottom-side rolling and/or sliding element 81. Furthermore, an element 82 which serves as a receiver for the rods, is embodied to be height-adjustable, for example via a screw connection to the base of the rolling and/or sliding element 81. As a result, the height in relation to the unit 84 can be adjusted, or it can be ensured that an x,y plane can be oriented parallel to a screen plane of the surgical set 3, and thus parallel to the implants 2. The platform, together with the image capturing equipment 4 arranged thereon, can also be displaced, in this case namely in a y direction, along the three rods which connect the units 84, 85 by means of a drive that engages the unit 84. The image capturing equipment 4 can thus travel over an entire x,y plane, and therefore also the screen plane located thereunder in a parallel position.

In FIG. 3, a connected evaluation unit 5, a data carrier 6, and a storage unit 7 are also illustrated. The corresponding components are typically integrated in a single device, but can also be present such that they are physically separate from one another.

In FIG. 2 and FIG. 3, the device 1 is illustrated by way of example with the aid of a construction. In practice, all components of the device 1 that are necessary for the measurement as such are integrated in a housing that is, to the extent necessary, open or openable at the bottom for the capture of images using the image capturing equipment 4, or is at least designed to be sufficiently transparent for the capture of images. This housing is, in terms of the dimensions of the bottom side, embodied such that the housing and the device 1 can be placed onto the surgical set 3 in a form fit. Preferably, not only is the bottom side of the housing embodied with a corresponding fit for this purpose, but rather the housing has an essentially cuboid shape or form. Handle pieces or notches can be provided on the sides so that the housing can be easily placed onto the surgical set 3 and removed therefrom again. The operating device can be arranged on the top side of the device 1, or can be attached thereon or integrated therein. In this case, an easy-to-handle device 1 is then ensured which is simply placed onto the surgical set 3 before the surgery. A scan that will be explained in greater detail below then occurs. Once this scan is finished, the housing is removed again, and the surgery can take place. Once the surgery is complete, the housing or the device 1 is placed again in order to perform another scan.

To perform the scan, the device 1 or the housing thereof is placed onto the surgical set 3 in a form fit, as can be seen schematically in FIG. 4. The device 1 can thus also be embodied as a lid, preferably as a lid with a height of less than 20 cm, in particular 10 cm or less.

A method according the invention is carried out in the following manner: A surgical set 3 having a plurality of implants 2 is provided for a specific surgery. The housing or the device 1 is then placed onto the surgical set 3. A scan of the implants 2 occurs. Each individual implant is thereby identified in that a plurality of images is captured during the scan. These images are evaluated in particular in regard to batch numbers and/or part numbers. An evaluation can thereby take place by means of image recognition, wherein individual digits and/or letters are recognized and possibly matched with stored digits and/or letters and, where necessary, also manufacturer information. Once a batch number or part number has been completely recognized, it is possible to switch from one implant 2 to the next. If an implant 2 is not recognized, this is outputted on the operating device. A user must then acknowledge that the corresponding part is not permitted to be used for the surgery due to lack of identification.

When the scan is complete, the device can be removed. The surgical set 3 is then available for a surgery. After the surgery, the device is once again placed onto the surgical set 3 and a scan takes place again, this time only a scan of the implants 2 as such, however, but not an additional recognition of any structural features of the implants 2. A difference is then generated from the scan that was performed prior to the surgery and the scan that was performed after the surgery. The implants 2 that were used or implanted during the surgery follow from the difference. Since these had previously been uniquely identified in the first scan, it is then also known which implants the patient being treated has had implanted.

1. A method for documenting a use of at least one implant used in a surgery and/or for the localization thereof, wherein the implant can be provided for a surgery and used in the surgery, comprising:
a) providing a surgical set having a plurality of implants;
b) capturing a first sequence of images of the plurality of implants of the surgical set using a device, wherein the surgical set is illuminated at least in the region of one implant using light from a light source during the capture of the sequence of images, wherein the operation set is scanned with an image recording device of the device and at the same time with the light source;
c) analyzing the sequence of images of the plurality of implants in order to identify each individual implant;
d) optionally outputting a signal when one and/or each implant has been identified;
e) capturing a second sequence of images of the plurality of implants of the surgical set using the device after a surgery in order to ascertain missing implants;
f) classifying a missing implant as used in surgery.

2. The method according to claim 1, wherein in b) the surgical set is traveled over by image capturing equipment of the device.

3. The method according to claim 2, wherein an enlarged image is outputted by the image capturing equipment.

4. The method according to claim 2, wherein the image capturing equipment is essentially guided over the surgical set in a plane.

5. (canceled)

6. The method according to claim 1, wherein the identification in c) is carried out via a batch number and/or part number read out from the images.

7. The method according to claim 1, wherein the identification in c) includes an identification of structural features.

8. The method according to claim 6, wherein the batch number and/or part number and/or structural features are determined from captured images and matched with a database.

9. The method according to claim 8, wherein b) and c) are repeated if not all implants are identified.

10. The method according to claim 1, wherein a surgical set having a screen is provided, and wherein the implants are arranged on and/or attached to the screen.

11. The method according to claim 1, wherein an audible and/or a visual signal is outputted if all implants have been uniquely identified.

12. A device for documenting and/or localizing a use of at least one implant used in a surgery, wherein the implant can be provided for a surgery and used in the surgery, comprising image capturing equipment for capturing a sequence of images of implants of a surgical set, wherein a light source for illuminating the implants during the capture of the sequence of images is provided and wherein the image recording device can be moved, evaluation equipment, and optionally a data carrier on which a dataset is stored, wherein the sequence of images can be evaluated using the evaluation equipment in order to identify implants, and a storage unit via which the localization of an implant can be depicted.

13. The device according to claim 12, wherein a movement mechanism is provided with which the image capturing equipment can be moved.

14. The device according to claim 13, wherein the image capturing equipment can be moved in a plane using the movement mechanism.

15. (canceled)
